(19) United States
(12) Patent Application Publication
(10) Pub. No.: US 2021/0386348 A1
(43) Pub. Date: Dec. 16, 2021

(54) SMART TEXTILE SUITABLE FOR DETECTING MOVEMENT AND/OR DEFORMATION

(71) Applicant: INRIA INSTITUT NATIONAL DE RECHERCHE EN INFORMATIQUE ET EN AUTOMATIQUE, Le Chesnay (FR)

(72) Inventors: Ganna PUGACH, Talence (FR); David DANEY, Bordeaux (FR)

(21) Appl. No.: 17/291,601

(22) PCT Filed: Nov. 5, 2019
(86) PCT No.: PCT/EP2019/080214

§ 371 Date: May 5, 2021

(30) Foreign Application Priority Data
Nov. 6, 2018 (FR) 1860192

(51) Int. Cl. A61B 5/27; A61B 5/00; A61B 5/11; A61B 5/107; A61B 5/0536

(52) U.S. Cl. CPC A61B 5/27; A61B 5/6804; A61B 5/7267; A61B 5/458; A61B 2505/09; A61B 5/1116; A61B 5/1071; A61B 5/0536; A61B 5/4585

(57) ABSTRACT

A textile suitable for detecting movement and/or deformation comprises an electrically conductive fabric (8) which can expand in at least two directions, electrodes (12) arranged substantially regularly along the periphery of the fabric (8), a controller (14) designed to control the excitation of the electrodes (12) two by two according to a pattern such that all the electrodes (12) are successively excited, and to measure each time the voltage in the non-excited electrodes (12), and a computer (22) comprising a neural network inference engine and designed to receive the voltage measurements taken attire non-excited electrodes (12) in a given excitation cycle, in order to supply them to the neural network inference engine and to return a characteristic measurement of a movement having caused a deformation of the textile.

The invention relates to the field of so-called “smart” textiles.

Smart textiles are a subset of wearable technologies. The garments utilising these textiles enable the individuals wearing them to interact with the garment, to measure their physical activity, to remotely control their telephone, etc.

This is achieved by introducing computing, digital or electronic components, but also through the use of innovative polymer materials or chromic materials, or even conductive fibres and materials.

The majority of the applications of a smart garment are in fields such as health and sport. For example, BioSerenity offers a smart garment for detecting epileptic seizures. Cityzen Sciences offers smart garments for sportsmen and women. Using sensors placed on the textile, they measure activity and physiological data in real time.

Google and Levi's have collaborated to produce a smart jacket using a different approach based on the use of conductive threads. This jacket makes it possible to interact with a telephone through different types of contact with the sleeve.

Another possible application of smart garments is the capture of human movement and posture. The many solutions proposed are generally based on cameras and inertial sensors. These solutions have many disadvantages. For example, the solutions based on image analysis suffer from a large space requirement when they are precise, or from a high degree of imprecision when they are purely optical. Similarly, solutions based on inertial sensors have little precision and are complicated to calibrate.

Several interesting techniques have been proposed in robotics, such as Electrical Impedance Tomography (EIT) based on the reconstruction of the electric field at the surface of a conductive material. This non-invasive technique is already used in medical imaging in order to detect internal bodies by applying electrodes to the surface of a patient's skin and measuring the variations in the electric field.

Electrical Impedance Tomography (EIT) has been used in the articles of Kato et al. (“Tactile sensor without wire and sensing element in the tactile region based on eit method”, IEEE Sensors, pages 792-795, 2007), and of Yao and Soleimani (“A pressure mapping imaging device based on electrical impedance tomography of conductive fabrics”, Sensor Review, 32(4):310-317, 2012) in order to propose tactile sensors (pressure sensors). The articles of Nagakubo et al. (“A deformable and deformation sensitive tactile distribution sensor”, IEEE International Conference on Robotics and Biomimetics, ROBIO, pages 1301-1308, 2007), of Alirezaei et al. (“A highly stretchable tactile distribution sensor for smooth surfaced humanoids”, 7th IEEE-RAS International Conference on Humanoid Robots, pages 167-173, 2007 and “A tactile distribution sensor which enables stable measurement under high and dynamic stretch”, IEEE Symposium on 3D User Interfaces (3DUI), pages 87-93, 2009), and of Tawil et al. (“Improved image reconstruction for an eit-based sensitive skin with multiple internal electrodes”, IEEE Transactions on Robotics, 27(3):425-435, 2011), have proposed tactile devices of the “artificial skin” type for robots. Their approach consists in injecting current and measuring voltages using electrodes connected to the edges of a conductive fabric, then applying inverse problem analysis in order to reconstruct the local change in resistivity due to a pressure. Finally, the articles of Pugach et al. (“Electronic hardware design of a low cost tactile sensor device for physical human-robot interactions”, IEEE XXXIII International Scientific Conference Electronics and Nanotechnology, ELNANO, pages 445-449, 2013, “Neural learning of the topographic tactile sensory information of an artificial skin through a self-organizing map”, Advanced Robotics, 29(21):1393-1409, 2015, and “Touch-based admittance control of a robotic arm using neural learning of an artificial skin”, In 2016 IEEE/RSJ International Conference on Intelligent Robots and Systems (IROS), pages 3374-3380, 2016) describe the use of neural networks for reconstructing the distribution of resistance within a conductive film and locating pressure points.

All the applications of EIT to smart garments are therefore exclusively restricted to pressure measurement. No application exists which enables an extension to be measured.

However, none of these applications makes it possible to reliably measure activities such as the movement (for example the extension) of an arm, shoulder or knee. This type of measurement is particularly important in the context of health-related prevention, for example the prevention of musculoskeletal disorders (or MSD).

The present invention improves the situation. To this effect, the invention proposes a textile suitable for detecting movement and/or deformation, which comprises an electrically conductive fabric which can be extended in at least two directions, electrodes arranged substantially regularly along the periphery of the fabric, a controller designed to control the excitation of the electrodes, two by two, according to a pattern such that all the electrodes are successively excited, and to measure each time the voltage in the non-excited electrodes, and a calculator comprising a neural network inference engine and designed to receive the voltage measurements taken at the non-excited electrodes in a given excitation cycle, in order to supply them to the neural network inference engine and to return a characteristic measurement of a movement having caused a deformation of the textile.

Indeed, by applying the principles of the EIT method, it is possible to determine an articulation movement at the point where the extendible conductive fabric is placed, and thus to produce a smart garment for capturing human movements.

In various alternatives, the garment according to the invention can have one or more of the following features:
the controller is designed to excite the electrodes according to an EIT excitation pattern chosen from the group comprising an adjacent pattern, an opposite pattern and a transverse pattern,
the neural network interference engine undergoes training with measurements characteristic of a movement having caused a deformation of the textile and voltage measurements taken at the non-excited electrodes obtained according to the same excitation pattern as that applied by the controller,
the neural network has one perceptron,
the textile further comprises two demultiplexers and two multiplexers controlled by the controller in order to implement the excitation pattern of the electrodes and voltage measurements taken at the non-excited electrodes,
the textile further comprises a current source for generating the current used by the demultiplexers in order to excite the electrodes, and
the calculator is designed to return a joint measurement.

The invention also relates to a smart garment comprising a substantially electrically non-conductive fabric and a textile such as described above that is fixed on the fabric, but also a deformation sensor comprising a textile as described above.

Other features and advantages of the invention will become more apparent on reading the following description, taken from examples given by way of illustration and not being limiting, taken from the drawings, in which:

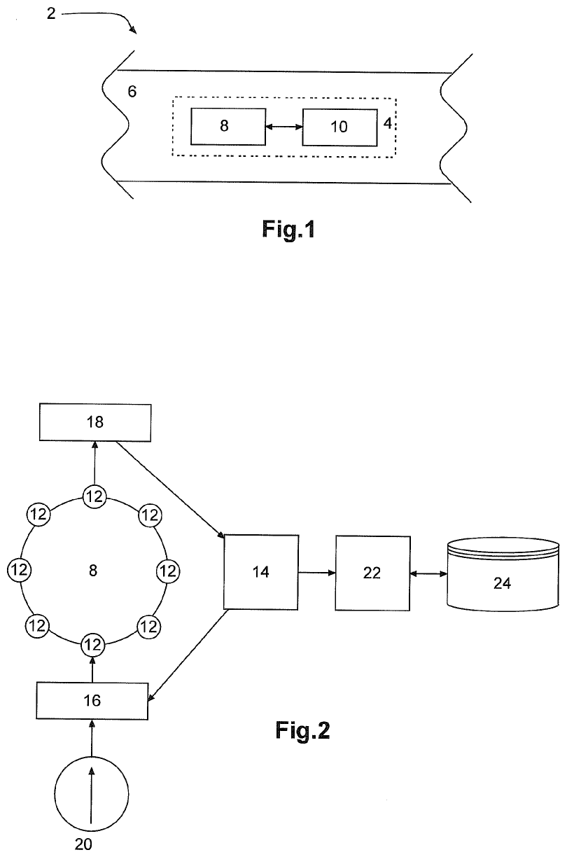

FIG. 1 is a schematic view of a garment incorporating a textile suitable for detecting movement according to the invention,

FIG. 2 is a schematic view of the textile suitable for detecting movement of FIG. 1, and

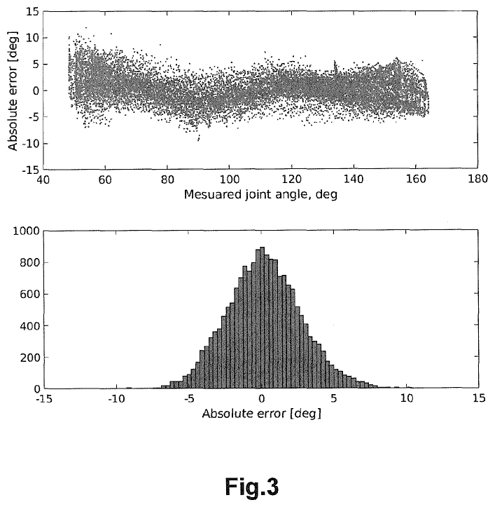

FIG. 3 shows the results of the angular measurement using the textile of the invention, and the actual error measured.

The drawings and the description below essentially contain elements of a certain nature. They can therefore not only serve as a means to better understand the present invention, but also contribute to its definition where appropriate.

FIG. 1 is a schematic view of a smart garment 2 incorporating a textile 4 suitable for detecting movement. The garment 2 comprises an electrically non-conductive or insulating fabric 6 on which the textile 4 is fixed. The fixing of the textile 4 on the fabric 6 can be carried out in any appropriate manner, including stitching, gluing, partial fusion or interweaving on the periphery of the textile 4.

The textile 4 comprises a fabric 8 and an extension measurement module 10. In the example described here, the fabric 8 is of the EeonTex type (registered trademark, fabric sold by Eeonyx under reference EeonTex LTT-SLPA), and is a conductive fabric that can extend in two directions, composed of 72% nylon and 28% spandex. It has a mass per unit area of approximately 162.72 g/m², a thickness of approximately 0.38 mm, an elongation recovery rate of 85% and a sheet resistance that can be adjusted by surface treatment between 10,000 ohms/inch² and 10,000,000 ohms/inch². Alternatively, any other extendible conductive fabric having the ability to extend in several directions (in other words, not having a preferred elongation direction such that the fabric substantially only extends in that preferred direction) can be used.

The fabric 8 is connected to a plurality of electrodes which are connected to the extension measurement module 10. FIG. 2 better illustrates the textile 4 and the relationship between the fabric 8 and the extension measurement module 10.

As can be seen in FIG. 2, the fabric 8 has a substantially circular shape and receives, in the example described here, eight electrodes 12 distributed substantially uniformly on the periphery of the fabric 8. The substantially circular shape is particularly suitable for carrying out measurements on an elbow or a knee. Alternatively, the textile 4 could comprise less electrodes, for example 4, or more electrodes, for example 16 or more.

The extension measurement module 10 comprises, in the example described here, a controller 14, two demultiplexers 16, two multiplexers 18, a source 20, a calculator 22 and a memory 24.

In the example described here, the controller 14 is an ARM 32-bit microcontroller (for example Atmel SAM3X8E ARM using a Cortex-M3 RISC processor). The controller 14 comprises a 12-bit analogue-to-digital converter for sampling the signals that it receives. The controller 14 has the role of controlling the demultiplexers 16 and the multiplexers 18 in order on the one hand to sequentially excite the electrodes 12 in pairs, by moving the ground each time, and on the other hand to measure the voltage drop at the terminals of the other electrodes 12.

The demultiplexers 16 and the multiplexers 18, in the example described here, are of the MAX306CPI+ type from Maxim Integrated. They respectively have the functions of demultiplexing excitation signals sent by the controller 14, and multiplexing measurement signals at the electrodes 12 as described above. From a functional point of view, the demultiplexers 16 and the multiplexers 18 can be considered to be coupled to the controller 14, to the extent that together they fulfil the function of excitation and measurement.

The controller 14 is designed to perform the excitation according to an EIT excitation pattern in order to induce, in the fabric 8, a change in resistance at the electrodes 12 which is characteristic of its extension. The current source 20 supplies the demultiplexers 16 with the current which is demultiplied for the excitation currents. The current source 20 can be DC, in which case the measurement voltage will be measured in the electrodes 12, or AC, in which case the amplitude and the offset of the voltage with respect to the AC current will be measured. Alternatively, the current source 20 could be omitted. In another alternative, the demultiplexers and the multiplexers could also be omitted by using a controller connected directly or indirectly to the electrodes.

EIT extension pattern shall mean a pattern chosen among:
an adjacent pattern, according to which the excitation current is introduced into neighbouring electrodes, and the voltage drop is measured successively in the other electrodes, each electrode pair being successively used in order to perform an excitation,
an opposite pattern, according to which the excitation current is introduced into diametrically opposed electrodes, and the voltage is measured successively in the other electrodes, each electrode pair being successively used in order to perform an excitation,
a transverse pattern, according to which the excitation current is introduced into electrodes which are opposed with respect to a fixed axis, and the voltage is measured successively in the other electrodes, each electrode pair being successively used in order to perform an excitation.

The work of the applicant has shown that the so-called “adjacent” pattern is the one which offers the best results. The work of the applicant suggests that this arises from the fact that this pattern offers a good compromise between sensitivity and selectivity.

The use of a microcontroller of the type described above makes it possible to keep the production costs modest, which enables the production of the textile 4 to be industrialised. Alternatively, the controller 14 could be replaced by another microcontroller or by a code executed by a processor. The term “processor” shall be understood as any processor suitable for the operations of the controller 14. Such a processor may be produced in any known manner, in the form of a microprocessor for a personal computer, a dedicated chip of the FPGA or SoC (system on chip) type, a computing resource on a grid, a microcontroller, or any other form suitable for supplying the computing power necessary for the embodiment described below. One or more of these elements can also be produced in the form of specialised electronic circuits such as an ASIC. A combination of processor and electronic circuits can also be envisaged.

In the example described here, the controller 14, the demultiplexers 16, the multiplexers 18 and the current source 20 make it possible to acquire data at a frequency of 45 Hz.

According to the EIT method, the current which passes through the fabric creates a volume distribution of the electric potential. The potential reduces along the current line as a function of distance with respect of the active electrodes between which the current is injected. The voltage drop per unit length (electric field intensity) is proportional to the intensity of the current and the resistance of the medium in accordance with Ohm's law. By measuring the voltage drop and by knowing the value of the current, the value of the resistance can then be calculated. A tomographic reconstruction algorithm enables the voltages, measured at the surface of the fabric only, to be used in order to calculate the spatial distribution of the resistivity inside the fabric.

Nevertheless, the model which describes the correlation between the posture/movement and the deformation of the fabric 8 is difficult to obtain analytically. For this reason, the applicant has had the idea of using the calculator 22 in combination with the memory 24.

The function of the calculator 22 is to apply the interference model of a neural network to the measurements of voltage drop at the electrodes 12. Hence, by training a neural network on the thousands of movements and corresponding voltage drop measurements, it becomes possible to do away with the determination of the model. More precisely, the applicant has discovered that an LMS network (for “Least Mean Squares”, for example a one perceptron neural network without softmax filtering at the output) used in the conditional learning paradigms, makes it possible to predict the joint angle with a precision of plus or minus 5 degrees.

FIG. 3 shows the results of the angular measurement using the textile of the invention, the actual error measured.

Here, the term “conditional learning paradigm” means an optimisation method which consists in the modification of synaptic weights of the neural network until the minimum mean quadratic error is found between the input and the desired output. Hence, by knowing the input joint angle, the learning of the neural network enables it to learn to predict a desired output derived from an unconditional stimulus and to associate this output with a conditional stimulus. This architecture is equivalent to a Pavlovian conditioning, which associates the prediction of a joint angle with the distribution of resistance in the fabric 8. By carrying out a learning according to an EIT excitation pattern, the learning thus allows the approach of this method to be faithfully reproduced. Furthermore, another type of neural network can be applied in order to associate the fabric extension with the joint angle, for example a supervised neural network or a convolutional neural network.

Moreover, the use of a neural network has the advantage of allowing a simpler, and therefore less expensive, calculator 22 to be used, since the application of a one perceptron neural network interference engine is much less calculation heavy than the resolution of the inverse problem of the conventional EIT method. In the example described here, the calculator 22 is a Raspberry-type computer or any other light computer with modest cost and which is suitable for implementing the one perceptron neural network interference engine with the voltage measurements originating from the controller 14. In the example described here, the calculator 22 is described in principle, and the memory 24 stores the one perceptron neural network interference engine, the voltage measurements originating from the controller 14, and the resulting angle measurements. The memory 24 may be any type of data storage suitable for receiving digital data: hard disk, solid-state drive (SSD), any form of flash memory, random-access memory, magnetic disk, locally distributed storage or storage in the cloud, etc. Preferably, it is embodied by the memory of the calculator 22.

Hence, the textile according to the invention:
is not intrusive and takes up little space,
is inexpensive in comparison with systems which use cameras,
is hardly limited by a capture visibility zone (which is often the case for external sensors), and is independent from occlusions (contrary to the systems which use cameras),
allows longitudinal measurements to be performed, contrary to systems with inertial cameras, which are subject to drift,
has no other sensor apart from the electrodes, and
is compatible with use in an industrial environment.

Moreover, thanks to the simplification of the EIT method through the use of neural networks, it is simple to implement and is not computer resources greedy. The use of neural networks also guarantees its scalability and the possibility of creating numerous profiles suitable for distinct measurements by training a plurality of interference engines specialised for the specific conditions.

The smart textile according to the invention can, more generally, be used in a deformation sensor which can be used to quantify the variation in the shape of a flexible object or of a multi-body articulated chain. For example, such a sensor could be used in a series robot, in soft robotics, for measuring the deformation of seats (aeronautic and automotive application), etc.

1. Textile suitable for detecting movement and/or deformation, characterised in that it comprises an electrically conductive fabric which can be extended in at least two directions, electrodes arranged substantially regularly along the periphery of the fabric, a controller designed to control the excitation of the electrodes, two by two, according to a pattern such that all the electrodes are successively excited, and to measure each time the voltage in the non-excited electrodes, and a calculator comprising a neural network inference engine and designed to receive the voltage measurements taken at the non-excited electrodes in a given excitation cycle, in order to supply them to the neural network inference engine and to return a characteristic measurement of a movement having caused a deformation of the textile.

2. Textile according to claim 1, wherein the controller is designed to excite the electrodes according to an EIT excitation pattern chosen from the group comprising an adjacent pattern, an opposite pattern and a transverse pattern.

3. Textile according to claim 1, wherein the neural network interference engine undergoes learning with measurements characteristic of a movement having caused a deformation of the textile and voltage measurements taken at the non-excited electrodes obtained according to the same excitation pattern as that applied by the controller.

4. Textile according to claim 3, wherein the neural network has one perceptron.

5. Textile according to claim 1, further comprising two demultiplexers and two multiplexers controlled by the controller in order to implement the excitation pattern of the electrodes and voltage measurements taken at the non-excited electrodes.

6. Textile according to claim 5, further comprising a current source for generating the current used by the demultiplexers in order to excite the electrodes.

7. Textile according to claim 1, wherein the calculator is designed to return a joint measurement.

8. Smart garment, comprising a substantially electrically non-conductive fabric and a textile according to claim 1 fixed on the fabric.

9. Deformation sensor, comprising a textile according to claim 1.
